(19) United States
(12) Patent Application Publication
(10) Pub. No.: US 2021/0102172 A1
(43) Pub. Date: Apr. 8, 2021

(54) REGENERATIVE ENDOTHELIAL PROGENITOR CELLS DERIVED FROM PLACENTAL SOURCES

(71) Applicant: WCTE Inc., Pinole, CA (US)

(72) Inventors: Thomas Ichim, San Diego, CA (US); Wes Chandler, Pinole, CA (US)

(73) Assignee: WCTE Inc., Pinole, CA (US)

(21) Appl. No.: 17/063,410

(22) Filed: Oct. 5, 2020

Related U.S. Application Data
(60) Provisional application No. 62/910,318, filed on Oct. 3, 2019.

(51) Int. Cl. C12N 5/0775; C12N 5/071

(52) U.S. Cl. CPC C12N 5/0668; C12N 5/0692; C12N 2506/025; C12N 2501/515; C12N 2501/165; C12N 2501/119; C12N 2501/115; C12N 2501/12; C12N 2501/135; C12N 2501/105; C12N 2501/11; C12N 2501/113

(57) ABSTRACT

Disclosed cell therapeutics useful for regenerative, immune modulatory and angiogenic applications. In one embodiment the invention teaches uses of placentally derived cells possessing endothelial and mesenchymal features, said cells obtained by enriching for a subpopulation of cells in which said subpopulation expresses a CD45 negative phenotypic profile and further enriching for cells that express which express CD56. Said cells may be modified by culture in conditions that enhance regenerative, immunological, or angiogenic activities.

### CROSS-REFERENCE TO RELATED APPLICATIONS

This application claims the benefit of priority to U.S. Provisional Application No. 62/910,318, filed Oct. 3, 2019, the contents of which are incorporated herein by reference.

### FIELD OF THE INVENTION

The invention pertains to the area of stem cell therapeutics, more specifically, the invention pertains to the area of mesenchymal stem cell therapeutics, more specifically, the invention pertains to means of selecting stem cells from placenta possessing placental efficacy compared to other stem cells, furthermore the invention pertains to the area of stem cell efficacy markers.

BACKGROUND

According to the definition by the U.S. Food and Drug Administration (FDA), somatic cell therapy (or cell therapy) is the prevention, treatment, cure, diagnosis, or mitigation of diseases or injuries in humans by the administration of autologous, allogeneic or xenogeneic cells that have been manipulated or altered ex vivo. Generally, said manipulation and alteration include the propagation, expansion, selection, and/or pharmacological treatment of the cells. The goal of cell therapy is to repair, replace or restore damaged tissues or organs. Cell therapy may provide extensive applications in modern medicine. For example, in Nov. 10, 2011, the U.S. FDA granted marketing approval to the New York Blood Center's allogeneic cord-blood product, HEMACORD, the first FDA-licensed hematopoietic progenitor cell therapy. HEMACORD is indicated for hematopoietic progenitor cell (HPC) transplantation procedures in patients with inherited, acquired, or myeloablative-treatment-related diseases that affect the hematopoietic system. Once the HPCs are infused into patients, the cells migrate to the bone marrow where they divide and mature. When the mature cells move into the bloodstream they can partially or fully restore the number and function of many blood cells, including immune function.

Mesenchymal stem cell therapeutics has entered the clinical arena in the treatment of various degenerative conditions including cardiovascular, neurological, and immunological. Regulatory approval of mesenchymal stem cell based products has been achieved in several jurisdictions, particularly of mesenchymal stem cells. Mesenchymal stem cells are classically defined as adherent cells possessing ability to differentiate into osteoblasts, adipocytes and chondrocytes and possessing the surface markers CD73, CD90, and CD105, while lacking the markers CD14, CD34, and CD45.

The current invention provides means of isolating, expanding, and clinically utilizing stem cells.

### SUMMARY

Embodiments herein are directed to mesenchymal stem cells and methods of generating mesenchymal stem cells possessing properties of endothelial progenitor cells comprising:
(i) isolating a mammalian cellular population;
(ii) enriching said mammalian cellular population for a subpopulation of cells expressing a CD45− phenotypic profile;
(iii) enriching said CD45− cells for a subpopulation of cells which express a CD56+ phenotypic profile; and
(iv) isolating the subpopulation of said CD56+ cells for a subpopulation of cells which express a phenotypic profile selected from the group consisting of: CD31+ and CD45−.

Further embodiments herein are directed to methods wherein said isolated subpopulation of cells which express a phenotypic profile selected from the group consisting of: CD31+ and CD45− are plastic adherent.

Further embodiments herein are directed to methods wherein said CD31+ cell population of cells is derived from fetal cells of the placenta.

Further embodiments herein are directed to methods wherein said CD31− and CD56+ cell populations are derived from fetal cells of the placenta.

Further embodiments herein are directed to methods wherein said CD31−, CD56+ cell, and CD45− cell populations are derived from fetal cells of the placenta.

Further embodiments herein are directed to methods wherein said endothelial progenitor cell property is ability to stimulate angiogenic activity.

Further embodiments herein are directed to methods wherein said angiogenic activity is associated with the ability to produce, in absence of stimulation cytokines, a factor selected from the group consisting of: a) VEGF, b) FGF-alpha, b) FGF-beta, c) FGF-5, d) HGF, e) PDGF, f) IGF, and g) EGF.

Further embodiments herein are directed to methods wherein said enhanced angiogenic activity is associated with ability to produce cytokines following activation selected from the group consisting of: a) VEGF, b) FGF-alpha, b) FGF-beta, c) FGF-5, d) HGF, e) PDGF, f) IGF, and g) EGF.

Further embodiments herein are directed to methods wherein said stimulation is exposure to an inflammatory signal.

Further embodiments herein are directed to methods wherein said inflammatory signal is an activator of a toll like receptor.

Further embodiments herein are directed to methods wherein said toll like receptor is TLR-1.

Further embodiments herein are directed to methods wherein said activator of TLR-1 is Pam3CSK4.

Further embodiments herein are directed to methods wherein said toll like receptor is TLR-2.

Further embodiments herein are directed to methods wherein said activator of TLR-2 is HKLM.

Further embodiments herein are directed to methods wherein said toll like receptor is TLR-3.

Further embodiments herein are directed to methods wherein said activator of TLR-3 is Poly:IC.

Further embodiments herein are directed to methods wherein said toll like receptor is TLR-4.

Further embodiments herein are directed to methods wherein said activator of TLR-4 is LPS.

Further embodiments herein are directed to methods wherein said activator of TLR-4 is Buprenorphine.

Further embodiments herein are directed to methods wherein said activator of TLR-4 is Carbamazepine.

### DESCRIPTION OF THE INVENTION

The invention teaches means of selecting mesenchymal stem cells (MSC) for placental efficacy based on expression of CD56, or lack of expression of certain proteins, said stem cells being isolated from placenta. In a specific embodiment, said mesenchymal stem cells are isolated so to possess substantial homogeneity and to be highly of fetal origin. Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation. Within the context of the current invention mesenchymal stem cells of fetal origin are extracted, or isolated to possess placental therapeutic efficacy, in part by selecting of stem cells that are primarily of fetal tissue origin.

As used herein, the phrase differentiates into a mesodermal, ectodermal or endodermal lineage refers to a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, chondrogenic, cardiogenic, dermatogenic, hematopoetic, hemangiogenic, myogenic, nephrogenic, urogenitogenic, osteogenic, pericardiogenic, or stromal. Examples of cells that differentiate into ectodermal lineage include, but are not limited to epidermal cells, neurogenic cells, and neurogliagenic cells. Examples of cells that differentiate into endodermal lineage include, but are not limited to, pleurigenic cells, hepatogenic cells, cells that give rise to the lining of the intestine, and cells that give rise to pancreogenic and splanchogenic cells.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes or at least supports, survival, growth, proliferation and/or maturation of a cell, or stimulates increased activity of a cell.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSCcan be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlioStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

Oct-4 (oct-3 in humans) is a transcription factor expressed in the pregastrulation embryo, early cleavage stage embryo, cells of the inner cell mass of the blastocyst, and embryonic carcinoma (“EC”) cells (Nichols, J. et al. (1998) Cell 95: 379-91), and is down-regulated when cells are induced to differentiate. The oct-4 gene (oct-3 in humans) is transcribed into at least two splice variants in humans, oct-3A and oct-3B. The oct-3B splice variant is found in many differentiated cells whereas the oct-3A splice variant (also previously designated oct-3/4) is reported to be specific for the undifferentiated embryonic stem cell. See Shimozaki et al. (2003) Development 130: 2505-12. Expression of oct-3/4 plays an important role in determining early steps in embryogenesis and differentiation. Oct-3/4, in combination with rox-1, causes transcriptional activation of the Zn-finger protein rex-1, which is also required for maintaining ES cells in an undifferentiated state (Rosfjord, E. and Rizzino, A. (1997) Biochem Biophys Res Commun 203: 1795-802; Ben-Shushan, E. et al. (1998) Mol Cell Biol 18: 1866-78). In some embodiments of the invention mesenchymal stem cells are selected for placental expression of OCT-4. In other embodiments, OCT-4 expression is used as a means of identifying cells for culture and expansion subsequent to exposure to various culture conditions.

Inflammatory conditions is an inclusive term and includes, for example: (1) tissue damage due to ischemia-reperfusion following acute myocardial infarction, aneurysm, stroke, hemorrhagic shock, crush injury, multiple organ failure, hypovolemic shock intestinal ischemia, spinal cord injury, and traumatic brain injury; (2) inflammatory disorders, e.g., burns, endotoxemia and septic shock, adult respiratory distress syndrome, cardiopulmonary bypass, hemodialysis; anaphylactic shock, severe asthma, angioedema, Crohn's disease, sickle cell anemia, poststreptococcal glomerulonephritis, membranous nephritis, and pancreatitis; (3) transplant rejection, e.g., hyperacute xenograft rejection; (4) pregnancy related diseases such as recurrent fetal loss and pre-eclampsia, and (5) adverse drug reactions, e.g., drug allergy, IL-2 induced vascular leakage syndrome and radiographic contrast media allergy. Complement-mediated inflammation associated with autoimmune disorders including, but not limited to, myasthenia gravis, Alzheimer's disease, multiple sclerosis, rheumatoid arthritis, systemic lupus erythematosus, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barre syndrome, Hashimoto's disease, idiopathic thrombocytopenic purpura, pemphigus, Sjogren's syndrome, and Takayasu's arteritis, may also be detected with the methods described herein.

Neurodegenerative condition (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, e.g. due to stroke, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

Mesenchymal stem cells (“MSC”) were originally derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2.sup.+ SH4.sup.+ CD29.sup.+ CD44.sup.+ CD71.sup.+ CD90.sup.+ CD106.sup.+ CD120a.sup.+ CD124.sup.+ CD14.sup.− CD34.sup.− CD45.sup.−)). The invention teaches the use of various mesenchymal stem cells

In a presently preferred embodiment, the isolation procedure also utilizes an enzymatic digestion process. Enzymes are used to dissociated tissue to extract cellular populations that are subsequently harvested and grown for isolation of fetal derived mesenchymal stem cells. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD45, CD117, CD141, or HLA-DR,DP, DQ.

In some embodiments of the invention MSC that are originally CD34 positive are genetically modified to possess placental neuromodulatory and neuroprotective properties. Said transfection may be accomplished by use of lentiviral vectors, said means to perform lentiviral mediated transfection are well-known in the art and discussed in the following references [1-7]. Some specific examples of lentiviral based transfection of genes into MSC include transfection of SDF-1 to promote stem cell homing, particularly hematopoietic stem cells [8]GDNF to treat Parkinson's in an animal model [9], HGF to accelerate remyelination in a brain injury model [10], akt to protect against pathological cardiac remodeling and cardiomyocyte death [11], TRAIL to induce apoptosis of tumor cells [12-15], PGE-1 synthase for cardioprotection [16], NUR77 to enhance migration [17], BDNF to reduce ocular nerve damage in response to hypertension [18], HIF-1 alpha to stimulate osteogenesis [19], dominant negative CCL2 to reduce lung fibrosis [20], interferon beta to reduce tumor progression [21], HLA-G to enhance immune suppressive activity [22], hTERT to induce differentiation along the hepatocyte lineage [23], cytosine deaminase [24], OCT-4 to reduce senescence [25, 26], BAMBI to reduce TGF expression and protumor effects [27], HO-1 for radioprotection [28], LIGHT to induce antitumor activity [29], miR-126 to enhance angiogenesis [30, 31], bcl-2 to induce generation of nucleus pulposus cells [32], telomerase to induce neurogenesis [33], CXCR4 to accelerate hematopoietic recovery [34] and reduce unwanted immunity [35], wnt11 to promote regenerative cytokine production [36], and the HGF antagonist NK4 to reduce cancer [37].

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14− and CD-45 positive cells. Cells were detached with 0.05% trypsin-EDTA , washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody . Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill, USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the invention MSC are transfected with anti-apoptotic proteins to enhance in vivo longevity. The present invention includes a method of using MSC that have been cultured under conditions to express increased amounts of at least one anti-apoptotic protein as a therapy to inhibit or prevent apoptosis. In one embodiment, the MSC which are used as a therapy to inhibit or prevent apoptosis have been contacted with an apoptotic cell. The invention is based on the discovery that MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MSC have been previously described which can be applied to the current invention, said antiapoptotic genes that can be utilized for practice of the invention, in a nonlimiting way, include GATA-4 [38], FGF-2 [39], bcl-2 [32, 40], and HO-1 [41]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the invention MSC are pretreated with agents to induce expression of antiapoptotic genes, one example is pretreatment with exendin-4 as previously described [42]. In a further non-limiting embodiment, MSC used in the present invention can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSC are cultured in a manner that provides MSC to express increased amounts of at least one anti-apoptotic protein. Culture conditions for growth of clinical grade MSC have been described in the literature and are incorporated by reference [43-76]

Without being limited to any one or more explanatory mechanisms for the immunomodulatory, regenerative and other properties, activities, and effects of MSC, it is worth nothing that they can modulate immune responses through a variety of modalities. For instance, MSC can have direct effects on a graft or host. Such direct effects are primarily a matter of direct contact between placental MSC and cells of the host or graft. The contact may be with structural members of the cells or with constituents in their immediate environment. Such direct mechanisms may involve direct contact, diffusion, uptake, or other processes well known to those skilled in the art. The direct activities and effects of the MSC may be limited spatially, such as to an area of local deposition or to a bodily compartment accessed by injection.

MSC also can “home” in response to “homing” signals, such as those released at sites of injury or disease. Since homing often is mediated by signals whose natural function is to recruit cells to the sites where repairs are needed, the homing behavior can be a powerful tool for concentrating Placental MSC to therapeutic targets. This effect can be stimulated by specific factors, as discussed below.

MSC may also modulate immune processes by their response to factors. This may occur additionally or alternatively to direct modulation. Such factors may include homing factors, mitogens, and other stimulatory factors. They may also include differentiation factors, and factors that trigger particular cellular processes. Among the latter are factors that cause the secretion by cells of other specific factors, such as those that are involved in recruiting cells, such as stem cells (including Placental MSC), to a site of injury or disease.

MSC may, in addition to the foregoing or alternatively thereto, secrete factors that act on endogenous cells, such as stem cells or progenitor cells. The factors may act on other cells to engender, enhance, decrease, or suppress their activities. MSC may secrete factors that act on stem, progenitor, or differentiated cells causing those cells to divide and/or differentiate. One such factor is exosomes and microvesicles produced by said placental MSC. MSC that home to a site where repair is needed may secrete trophic factors that attract other cells to the site. In this way, MSC may attract stem, progenitor, or differentiated cells to a site where they are needed. MSC also may secrete factors that cause such cells to divide or differentiate. Secretion of such factors, including trophic factors, can contribute to the efficacy of placental MSC in, for instance, limiting inflammatory damage, limiting vascular permeability, improving cell survival, and engendering and/or augmenting homing of repair cells to sites of damage. Such factors also may affect T-cell proliferation directly. Such factors also may affect dendritic cells, by decreasing their phagocytic and antigen presenting activities, which also may affect T-cell activity. Furthermore such factors, or MSC themselves, may be capable of modulating T regulatory cell numbers.

By these and other mechanisms, MSC can provide beneficial immunomodulatory effects, including, but not limited to, suppression of undesirable and/or deleterious immune reactions, responses, functions, diseases, and the like. MSC in various embodiments of the invention provide beneficial immunomodulatory properties and effects that are useful by themselves or in adjunctive therapy for precluding, preventing, lessening, decreasing, ameliorating, mitigating, treating, eliminating and/or curing deleterious immune processes and/or conditions. Such processes and conditions include, for instance, autoimmune diseases, anemias, neoplasms, HVG, GVHD, and certain inflammatory disorders. In one particular embodiment, said placental MSC are useful for treatment of Neurological disease, inflammatory conditions, psychiatric disorders, inborn errors of metabolisms, vascular disease, cardiac disease, renal disease, hepatic disease, pulmonary disease, ocular conditions such as uveitis, gastrointestinal disorders, orthopedic disorders, dermal disorders, neoplasias, prevention of neoplasias, hematopoietic disorders, reproductive disorders, gynecological disorders, urological disorders, immunological disorders, olfactory disorders, and auricular disorders.

In some embodiments the MSC preparations are clonally derived. In principle, the MSC in these preparations are genetically identical to one another and, if properly prepared and maintained, are free of other cells. In some embodiments MSC preparations that are less pure than these may be used. While rare, less pure populations may arise when the initial cloning step requires more than one cell. If these are not all MSC, expansion will produce a mixed population in which MSC are only one of at least two types of cells. More often mixed populations arise when MSC are administered in admixture with one or more other types of cells.

In many embodiments the purity of MSC for administration to a subject is about 100%. In other embodiments it is 95% to 100%. In some embodiments it is 85% to 95%. Particularly in the case of admixtures with other cells, the percentage of MSC can be 25%-30%, 30%-35%, 35%-40%, 40%-45%, 45%-50%, 60%-70%, 70%-80%, 80%-90%, or 90%-95%.

The number of MSC in a given volume can be determined by well known and routine procedures and instrumentation. The percentage of MSC in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

MSC immunomodulation may involve undifferentiated MSC, or MSC that have been dedifferentiated by treatment with agents such as valproic acid. It may involve MSC that are committed to a differentiation pathway. Such immunomodulation also may involve placental MSC that have differentiated into a less potent stem cell with limited differentiation potential. It also may involve MSC that have differentiated into a terminally differentiated cell type. The best type or mixture of MSC will be determined by the particular circumstances of their use, and it will be a matter of routine design for those skilled in the art to determine an effective type or combination of MSC.

The choice of formulation for administering MSC for a given application will depend on a variety of factors. Prominent among these will be the species of subject, the nature of the disorder, dysfunction, or disease being treated and its state and distribution in the subject, the nature of other therapies and agents that are being administered, the optimum route for administration of the placetnal MSC, survivability of MSC via the route, the dosing regimen, and other factors that will be apparent to those skilled in the art. In particular, for instance, the choice of suitable carriers and other additives will depend on the exact route of administration and the nature of the particular dosage form, for example, liquid dosage form (e.g., whether the composition is to be formulated into a solution, a suspension, gel or another liquid form, such as a time release form or liquid-filled form). ¶

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic MSC. Various embodiments of the invention comprise measures to increase cell survival and/or to overcome problems posed by barriers to seeding and/or growth. ¶

Examples of compositions comprising MSC include liquid preparations, including suspensions and preparations for intramuscular or intravenous administration (e.g., injectable administration), such as sterile suspensions or emulsions. Such compositions may comprise an admixture of MSC with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Compositions of the invention often are conveniently provided as liquid preparations, e.g., isotonic aqueous solutions, suspensions, emulsions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues.

Various additives often will be included to enhance the stability, sterility, and isotonicity of the compositions, such as antimicrobial preservatives, antioxidants, chelating agents, and buffers, among others. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents that delay absorption, for example, aluminum monostearate, and gelatin. According to the present invention, however, any vehicle, diluent, or additive used would have to be compatible with the cells.

Placetnal MSC solutions, suspensions, and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose) may also be present.

Typically, the compositions will be isotonic, i.e., they will have the same osmotic pressure as blood and lacrimal fluid when properly prepared for administration.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or cell stabilizer can be employed to increase the life of MSC compositions. If such preservatives are included, it is well within the purview of the skilled artisan to select compositions that will not affect the viability or efficacy of the MSC.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells utilized in practicing the present invention in the required amount of the appropriate solvent with various amounts of the other ingredients, as desired.

In some embodiments, MSC are formulated in a unit dosage injectable form, such as a solution, suspension, or emulsion. Pharmaceutical formulations suitable for injection of MSC typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

For any composition to be administered to an animal or human, and for any particular method of administration, it is preferred to determine therefore: toxicity, such as by determining the lethal dose (LD) and LD50 in a suitable animal model, e.g., rodent such as mouse or rat; and, the dosage of the composition(s), concentration of components therein, and timing of administering the composition(s), which elicit a suitable response. Such determinations do not require undue experimentation from the knowledge of the skilled artisan, this disclosure, and the documents cited herein. And, the time for sequential administrations can be ascertained without undue experimentation.

In some embodiments MSC are encapsulated for administration, particularly where encapsulation enhances the effectiveness of the therapy, or provides advantages in handling and/or shelf life. Encapsulation in some embodiments where it increases the efficacy of MSC mediated immunosuppression may, as a result, also reduce the need for immunosuppressive drug therapy.

Also, encapsulation in some embodiments provides a barrier to a subject's immune system that may further reduce a subject's immune response to the Placental MSC (which generally are not immunogenic or are only weakly immunogenic in allogeneic transplants), thereby reducing any graft rejection or inflammation that might occur upon administration of the cells.

In a variety of embodiments where placental MSC are administered in admixture with cells of another type, which are more typically immunogenic in an allogeneic or xenogeneic setting, encapsulation may reduce or eliminate adverse host immune responses to the non-placental MSC cells and/or GVHD that might occur in an immunocompromised host if the admixed cells are immunocompetent and recognize the host as non-self.

Placental MSC may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval.

A wide variety of materials may be used in various embodiments for microencapsulation of Placental MSC. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of Placental MSC are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of Placental MSC.

Certain embodiments incorporate Placental MSC into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, Placental MSC may be incorporated in the interstices of a three-dimensional gel. A large polymer or gel, typically, will be surgically implanted. A polymer or gel that can be formulated in small enough particles or fibers can be administered by other common, more convenient, non-surgical routes.

Pharmaceutical compositions of the invention may be prepared in many forms that include tablets, hard or soft gelatin capsules, aqueous solutions, suspensions, and liposomes and other slow-release formulations, such as shaped polymeric gels. Oral liquid pharmaceutical compositions may be in the form of, for example, aqueous or oily suspensions, solutions, emulsions, syrups, or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives. An oral dosage form may be formulated such that cells are released into the intestine after passing through the stomach. Such formulations are described in U.S. Pat. No. 6,306,434 and in the references contained therein.

Pharmaceutical compositions suitable for rectal administration can be prepared as unit dose suppositories. Suitable carriers include saline solution and other materials commonly used in the art.

For administration by inhalation, cells can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, a means may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, cells may be administered via a liquid spray, such as via a plastic bottle atomizer.

Placental MSC may be administered with other pharmaceutically active agents. In some embodiments one or more of such agents are formulated together with Placental MSC for administration. In some embodiments the Placental MSC and the one or more agents are in separate formulations. In some embodiments the compositions comprising the Placental MSC and/or the one or more agents are formulated with regard to adjunctive use with one another.

Placental MSC may be administered in a formulation comprising a immunosuppressive agents, such as any combination of any number of a corticosteroid, cyclosporin A, a cyclosporin-like immunosuppressive agent, cyclophosphamide, antithymocyte globulin, azathioprine, rapamycin, FK-506, and a macrolide-like immunosuppressive agent other than FK-506 and rapamycin. In certain embodiments, such agents include a corticosteroid, cyclosporin A, azathioprine, cyclophosphamide, rapamycin, and/or FK-506. Immunosuppressive agents in accordance with the foregoing may be the only such additional agents or may be combined with other agents, such as other agents noted herein. Other immunosuppressive agents include Tacrolimus, Mycophenolate mofetil, and Sirolimus.

Such agents also include antibiotic agents, antifungal agents, and antiviral agents, to name just a few other pharmacologically active substances and compositions that may be used in accordance with embodiments of the invention.

Typical antibiotics or anti-mycotic compounds include, but are not limited to, penicillin, streptomycin, amphotericin, ampicillin, gentamicin, kanamycin, mycophenolic acid, nalidixic acid, neomycin, nystatin, paromomycin, polymyxin, puromycin, rifampicin, spectinomycin, tetracycline, tylosin, zeocin, and cephalosporins, aminoglycosides, and echinocandins.

Further additives of this type relate to the fact that Placental MSC, like other stems cells, following administration to a subject may “home” to an environment favorable to their growth and function. Such “homing” often concentrates the cells at sites where they are needed, such as sites of immune disorder, dysfunction, or disease. A number of substances are known to stimulate homing. They include growth factors and trophic signaling agents, such as cytokines. They may be used to promote homing of Placental MSC to therapeutically targeted sites. They may be administered to a subject prior to treatment with Placental MSC, together with placental MSC, or after placental MSC are administered.

Certain cytokines, for instance, alter or affect the migration of placental MSC or their differentiated counterparts to sites in need of therapy, such as immunocompromised sites. Cytokines that may be used in this regard include, but are not limited to, stromal cell derived factor-1 (SDF-1), stem cell factor (SCF), angiopoietin-1, placenta-derived growth factor (PIGF), granulocyte-colony stimulating factor (G-CSF), cytokines that stimulate expression of endothelial adhesion molecules such as ICAMs and VCAMs, and cytokines that engender or facilitate homing.

They may be administered to a subject as a pre-treatment, along with Placental MSC, or after placental MSC have been administered, to promote homing to desired sites and to achieve improved therapeutic effect, either by improved homing or by other mechanisms. Such factors may be combined with Placental MSC in a formulation suitable for them to be administered together. Alternatively, such factors may be formulated and administered separately.

Order of administration, formulations, doses, frequency of dosing, and routes of administration of factors (such as the cytokines discussed above) and Placental MSC generally will vary with the disorder or disease being treated, its severity, the subject, other therapies that are being administered, the stage of the disorder or disease, and prognostic factors, among others. General regimens that have been established for other treatments provide a framework for determining appropriate dosing in placental MSC-mediated direct or adjunctive therapy. These, together with the additional information provided herein, will enable the skilled artisan to determine appropriate administration procedures in accordance with embodiments of the invention, without undue experimentation.

Placental MSC can be administered to a subject by any of a variety of routes known to those skilled in the art that may be used to administer cells to a subject.

Among methods that may be used in this regard in embodiments of the invention are methods for administering placental MSC by a parenteral route. Parenteral routes of administration useful in various embodiments of the invention include, among others, administration by intravenous, intraarterial, intracardiac, intraspinal, intrathecal, intraosseous, intraarticular, intrasynovial, intracutaneous, intradermal, subcutaneous, and/or intramuscular injection. In some embodiments intravenous, intraarterial, intracutaneous, intradermal, subcutaneous and/or intramuscular injection are used. In some embodiments intravenous, intraarterial, intracutaneous, subcutaneous, and/or intramuscular injection are used.

In various embodiments of the invention placental MSC are administered by systemic injection. Systemic injection, such as intravenous injection, offers one of the simplest and least invasive routes for administering placental MSC. In some cases, these routes may require high placental MSC doses for optimal effectiveness and/or homing by the placental MSC to the target sites. In a variety of embodiments placental MSC may be administered by targeted and/or localized injections to ensure optimum effect at the target sites.

Placental MSC may be administered to the subject through a hypodermic needle by a syringe in some embodiments of the invention. In various embodiments, placental MSC are administered to the subject through a catheter. In a variety of embodiments, placental MSC are administered by surgical implantation. Further in this regard, in various embodiments of the invention, Placental MSC are administered to the subject by implantation using an arthroscopic procedure. In some embodiments Placental MSC are administered to the subject in or on a solid support, such as a polymer or gel. In various embodiments, Placental MSC are administered to the subject in an encapsulated form.

In additional embodiments of the invention, Placental MSC are suitably formulated for oral, rectal, epicutaneous, ocular, nasal, and/or pulmonary delivery and are administered accordingly.

Compositions can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of placental MSC appropriate to be used in accordance with various embodiments of the invention will depend on numerous factors. It may vary considerably for different circumstances. The parameters that will determine optimal doses of placental MSC to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the Placental MSC are syngeneic, autologous, allogeneic, or xenogeneic; their potency (specific activity); the site and/or distribution that must be targeted for the Placental MSC to be effective; and such characteristics of the site such as accessibility to Placental MSC and/or engraftment of Placental MSC. Additional parameters include co-administration with Placental MSC of other factors (such as growth factors and cytokines). The optimal dose in a given situation also will take into consideration the way in which the cells are formulated, the way they are administered, and the degree to which the cells will be localized at the target sites following administration. Finally, the determination of optimal dosing necessarily will provide an effective dose that is neither below the threshold of maximal beneficial effect nor above the threshold where the deleterious effects associated with the dose of Placental MSC outweighs the advantages of the increased dose.

The optimal dose of placental MSC for some embodiments will be in the range of doses used for autologous, mononuclear bone marrow transplantation. For fairly pure preparations of placental MSC, optimal doses in various embodiments will range from 10.sup.4 to 10.sup.8 placental MSC cells/kg of recipient mass per administration. In some embodiments the optimal dose per administration will be between 10.sup.5 to 10.sup.7 placental MSC cells/kg. In many embodiments the optimal dose per administration will be 5.times.10.sup.5 to 5.times.10.sup.6 placental MSC cells/kg. By way of reference, higher doses in the foregoing are analogous to the doses of nucleated cells used in autologous mononuclear bone marrow transplantation. Some of the lower doses are analogous to the number of CD34.sup.+ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations.

In various embodiments, Placental MSC, placental MSC possessing EPC properties, also possessing expression of CD56, may be administered in an initial dose, and thereafter maintained by further administration of Placental MSC. Placental MSC may be administered by one method initially, and thereafter administered by the same method or one or more different methods. The subject's PLACENTAL MSC levels can be maintained by the ongoing administration of the cells. Various embodiments administer the Placental MSC either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regiments can be ascertained by the skilled artisan, from this disclosure, the documents cited herein, and the knowledge in the art.

The dose, frequency, and duration of treatment will depend on many factors, including the nature of the disease, the subject, and other therapies that may be administered. Accordingly, a wide variety of regimens may be used to administer Placental MSC.

In some embodiments Placental MSC are administered to a subject in one dose. In others Placental MSC are administered to a subject in a series of two or more doses in succession. In some other embodiments wherein Placental MSC are administered in a single dose, in two doses, and/or more than two doses, the doses may be the same or different, and they are administered with equal or with unequal intervals between them.

Placental MSC may be administered in many frequencies over a wide range of times. In some embodiments, placental MSC are administered over a period of less than one day. In other embodiment they are administered over two, three, four, five, or six days. In some embodiments Placental MSC are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

The immunomodulatory properties of placental MSC may be used in treating a wide variety of disorders, dysfunctions and diseases, such as those that, intrinsically, as a secondary effect or as a side effect of treatment, present with deleterious immune system processes and effects. Several illustrations are discussed below.

Many embodiments in this regard involve administering Placental MSC to a subject having a weakened (or compromised) immune system, either as the sole therapy or as adjunctive therapy with another treatment. In a variety of embodiments in this regard Placental MSC are administered to a subject adjunctively to radiation therapy or chemotherapy or a combination of radiation and chemotherapies that either have been, are being, or will be administered to the subject. In many such embodiments, the radiation therapy, chemotherapy, or a combination of radiation and chemotherapies are part of a transplant therapy. And in a variety of embodiments Placental MSC are administered to treat a deleterious immune response, such as HVG or GVHD.

In a variety of embodiments in this regard, the subject is the recipient of a non-syngeneic, typically allogeneic, blood cell or bone marrow cell transplant, the immune system of the subject has been weakened or ablated by radiation therapy, chemotherapy, or a combination of radiation and chemotherapy, immunosuppressive drugs are being administered to the subject, the subject is at risk to develop or has developed graft versus host disease, and Placental MSC are administered to the subject adjunctively to any one or more of the transplant, the radiation therapy and/or the chemotherapy, and the immunosuppressive drugs to treat, such as ameliorate, arrest, or eliminate, graft versus host disease in the subject.

In various embodiments, Placental MSC are administered to a subject suffering from a neoplasm, adjunctive to a treatment thereof. For example, in some embodiments of the invention in this regard, the subject is at risk for or is suffering from a neoplasm of blood or bone marrow cells and has undergone or will undergo a blood or bone marrow transplant. Using the methods described herein for placental MSC isolation, characterization, and expansion, together with the disclosures herein on immune-suppressing properties of Placental MSC, placental MSC are administered to treat, such as to prevent, suppress, or diminish, the deleterious immune reactions, such as HVG and GVHD, that may complicate the transplantation therapy.

In a variety of embodiments involving transplant therapies, placental MSC can be used alone for an immunosuppressive purpose, or together with other agents. Placental MSC can be administered before, during, or after one or more transplants. If administered during transplant, placental MSC can be administered separately or together with transplant material. If separately administered, the Placental MSC can be administered sequentially or simultaneously with the other transplant materials. Furthermore, Placental MSC may be administered well in advance of the transplant and/or well after, alternatively to or in addition to administration at or about the same time as administration of the transplant.

Other agents that can be used in conjunction with placental MSC, in transplantation therapies in particular, include immunomodulatory agents, such as those described elsewhere herein, particularly immunosuppressive agents, more particularly those described elsewhere herein, especially in this regard, one or more of a corticosteroid, cyclosporin A, a cyclosporin-like immunosuppressive compound, azathioprine, cyclophosphamide, methotrexate, and an immunosuppressive monoclonal antibody agent.

Among neoplastic disorders of bone marrow that are treated with Placental MSC in embodiments of the invention in this regard are myeloproliferative disorders (“MPDs”); myelodysplastic syndromes (or states) (“MDSs”), leukemias, and lymphoproliferative disorders including multiple myeloma and lymphomas.

MPDs are distinguished by aberrant and autonomous proliferation of cells in blood marrow. The disorder may involve only one type of cell or several. Typically, MPDs involve three cell lineages and are erythrocytic, granulocytic, and thrombocytic. Involvement of the three lineages varies from one MPD to another and between occurrences of the individual types. Typically, they are differently affected and one cell lineage is affected predominately in a given neoplasm. MPDs are not clearly malignant; but, they are classified as neoplasms and are characterized by aberrant, self-replication of hematopoietic precursor cells in blood marrow. MPDs have the potential, nonetheless, to develop into acute leukemias.

Placental MSC can modulate immune responses. In particular in this regard, it has been found that Placental MSC can suppress immune responses, including but not limited to immune responses involved in, for example, HVG response and GVHD, to name just two. In an even more detailed particular in this regard, it has been found that Placental MSC can suppress proliferation of T-cells, even in the presence of potent T-cell stimulators, such as Concanavalin A and allogeneic or xenogeneic stimulator cells.

Moreover, it has been found that even relatively small amounts of placental MSC can suppress these responses. Indeed, only 3% Placental MSC in mixed lymphocyte reactions is sufficient to reduce T-cell response by 50% in vitro.

In one embodiment of the invention, reduced numbers of placental MSC in a patient is used as a diagnostic for predisposition to degenerative disorders.

Accordingly, embodiments of the invention provide compositions and methods and the like for treating, such as for ameliorating, and/or curing or eliminating, neoplasms, such as neoplasms of hematopoietic cells, particularly those of bone marrow.

Embodiments of the invention relate to using placental MSC immunomodulation to treat an immune dysfunction, disorder, or disease, either solely, or as an adjunctive therapy. Embodiments in this regard relate to congenital immune deficiencies and autoimmune dysfunctions, disorders, and diseases. Various embodiments relate, in this regard, to using Placental MSC to treat, solely or adjunctively, Crohn's disease, Guillain-Barre syndrome, lupus erythematosus (also called “SLE” and systemic lupus erythematosus), multiple sclerosis, myasthenia gravis, optic neuritis, psoriasis, rheumatoid arthritis, Graves' disease, Hashimoto's disease, Ord's thyroiditis, diabetes mellitus (type 1), Reiter's syndrome, autoimmune hepatitis, primary biliary cirrhosis, antiphospholipid antibody syndrome (“APS”), opsoclonus-myoclonus syndrome (“OMS”), temporal arteritis, acute disseminated encephalomyelitis (“ADEM” and “ADE”), Goodpasture's, syndrome, Wegener's granulomatosis, celiac disease, pemphigus, polyarthritis, autism, autism spectrum disorder, post traumatic stress disorder, and warm autoimmune hemolytic anemia.

Particular embodiments among these relate to Crohn's disease, lupus erythematosus (also called “SLE” and systemic lupus erythematosus), multiple sclerosis, myasthenia gravis, psoriasis, rheumatoid arthritis, Graves' disease, Hashimoto's disease, diabetes mellitus (type 1), Reiter's syndrome, primary biliary cirrhosis, celiac disease, polyarhritis, and warm autoimmune hemolytic anemia.

The placental mesenchymal stem cells possessing EPC properties, in one embodiment are a population of cells comprising cells having the surface marker CD44 [77], cells having the surface marker CD13 [78, 79], cells having the surface marker CD90 [77, 80-85], cells having the surface marker CD105 [78, 81, 86-92], cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, VEGFR and cells having the surface marker Tie-2s. Said MSC possessing EPC properties may be treated in a manner to mimic the tumor microenvironment, this endows enhanced angiogenic activity as compared MSC EPC cultured under normal conditions. In one embodiment the cells are grown under the acidic conditions in the tumor microenvironment and are incorporated by reference [93-106]. In one embodiment of the invention, endothelial progenitor cells, or products thereof, are cultured under conditions in GCN2 kinase is activated [107, 108], said conditions include culture in the presence of uncharged tRNA [109-112], tryptophan deprivation [113-115],arginine deprivation [116-121], asparagine deprivation [122-126], and glutamine deprivation [127, 128].

In addition, placental MSC are used in a variety of embodiments in this regard, solely and, typically, adjunctively, to treat a variety of diseases thought to have an autoimmune component, including but not limited to embodiments that may be used to treat endometriosis, interstitial cystitis, neuromyotonia, scleroderma, progressive systemic scleroderma, vitiligo, vulvodynia, Chagas' disease, sarcoidosis, chronic fatigue syndrome, and dysautonomia. Inherited immune system disorders include Severe Combined Immunodeficiency (SCID) including but not limited to SCID with Adenosine Deaminase Deficiency (ADA-SCID), SCID which is X-linked, SCID with absence of T & B Cells, SCID with absence of T Cells, Normal B Cells, Omenn Syndrome, Neutropenias including but not limited to Kostmann Syndrome, Myelokathexis; Ataxia-Telangiectasia, Bare Lymphocyte Syndrome, Common Variable Immunodeficiency, DiGeorge Syndrome, Leukocyte Adhesion Deficiency; and phagocyte Disorders (phagocytes are immune system cells that can engulf and kill foreign organisms) including but not limited to Chediak-Higashi Syndrome, Chronic Granulomatous Disease, Neutrophil Actin Deficiency, Reticular Dysgenesis. Placental MSC may be administered adjunctively to a treatment for any of the foregoing diseases.

Various sources of MSC can be used which are considered placental MSC, these include placental sources, cord tissue, and cord blood. MSCs can also be cultured in vitro to maintain a source of MSC, or can be induced to produce further differentiated EPCs, the EPC are selected based on expression of CD56. The cells of the invention can be obtained by mechanically and enzymatically dissociating cells from placental, adipose, or umbilical cord tissue. Mechanical dissociation can be brought about using methods that include, without limitation, chopping and/or mincing the tissue, and/or centrifugation and the like. Enzymatic dissociation of connective tissue and from cell-to-cell associations can be brought about by enzymes including, but not limited to, Blendzyme, DNAse I, collegenase and trypsin, or a cocktail of enzymes found to be effective in liberating cells from the placenta sample. The procedure for mechanically and enzymatically isolating a cell of the present invention should not be construed to be limited to the materials and techniques presented herein, but rather it will be recognized that these techniques are well-established and fall well within the scope of experimental optimization performed routinely in the art. In the case of placenta-derived EPCs of the invention are isolated from placenta. In the isolation of the cells of the invention, placenta can be obtained from any animal by any suitable method. A first step in any such method requires the isolation of placenta from the source animal. The animal can be alive or dead, so long as cells within placenta are viable. Typically, human placenta is obtained from a living donor, using well-recognized surgical protocols. The cells of the invention are present in the initially excised or extracted placenta, regardless of the method by which placenta is obtained. In another embodiment, placenta may be obtained from non-human animals. In one embodiment, a placenta is removed from the animal. In one embodiment, placenta is washed with a physiologically-compatible solution, such as phosphate buffer saline (PBS). The washing step consists of rinsing placenta with PBS, agitating the tissue, and allowing the tissue to settle. In one embodiment, placenta is dissociated. The dissociation can occur by enzyme degradation and neutralization. Alternatively, or in conjunction with such enzymatic treatment, other dissociation methods can be used such as mechanical agitation, sonic energy, or thermal energy. In some instances, it may be desirable to further process the dissociated tissue. For example, the dissociated placenta is then filtered to isolate cells from other connective tissue. The extracted cells can be concentrated into a pellet. One method to concentrate the cells includes centrifugation, wherein the sample is centrifuged and the pellet retained. The pellet includes the placenta derived EPCs of the invention.

In one embodiment, the cells are resuspended and can be washed (e.g. in PBS). Cells can be centrifuged and resuspended successive times to achieve a greater purity. In one embodiment, the cells extracted from placenta may be a heterogeneous population of cell which includes the Placenta-derived EPCs of the invention. placenta -derived EPCs may be separated from other cells by methods that include, but are not limited to, cell sorting, size fractionation, granularity, density, molecularity, morphologically, and immunohistologically. In one embodiment, Placenta-derived EPCs of the invention are separated from other cells by assaying the length of the telomere, as stem cells tend to have longer telomeres compared to differentiated cells. In another embodiment, placenta-derived EPCs of the invention are separated from other cells by assaying telomeric activity, as telomeric activity can serve as a stem-cell specific marker. In another embodiment, placenta -derived endothelial cells of the invention are separated from other cells immunohistochemically, for example, by panning, using magnetic beads, or affinity chromatography.

In one embodiment tissue culture supernatant is derived from cultures of placental MSC and utilized for therapeutic applications. Use of tissue culture supernatant is described in the following patents and incorporated by reference U.S. Pat. Nos. 8,703,710; 9,192,632; 6,642,048; 7,790,455; 9,192,632; and the following patent applications; 20160022738; 20160000699; 20150024483; 20130251670; 20120294949; 20120276215; 20120195969; 20110293583; 20110171182; 20110129447; 20100159588; 20080241112.

Various aspects of the invention of the invention relating to the above are enumerated in the following paragraphs:

Aspect 1. A mesenchymal stem possessing enhanced therapeutic activity compared to conventional mesenchymal stem cells, wherein said mesenchymal stem cells with enhanced therapeutic activity are derived by purification for lack of expression of CD45 but express CD31.

Aspect 2. The mesenchymal stem cell of claim 1, wherein said stem cell is isolated by a method comprising the steps of: (i) isolating a mammalian cellular population; (ii) enriching for a subpopulation of the cells of step (i), which subpopulation expresses a CD45− phenotypic profile; and (a) enriching for a subpopulation of the CD45− cells derived from step (ii) which express a CD56+ phenotypic profile and isolating the subpopulation of said CD56+ cells which express a CD31+ phenotypic profile and/or (b) isolating the subpopulation of CD45− cells derived from step (ii) which express a CD34-phenotypic profile, to thereby isolate said mesenchymal stem cells with placental therapeutic activity as compared to conventional mesenchymal stem cells.

Aspect 3. The mesenchymal stem cell of claim 2, wherein said CD31+ cell population is derived from fetal cells of the placenta.

Aspect 4. The mesenchymal stem cells of claim 2, wherein said CD31−, CD56+ cell population is derived from fetal cells of the placenta.

Aspect 5. The mesenchymal stem cell of claim 2, wherein said CD31−, CD56+ cell, CD45− population is derived from fetal cells of the placenta.

Aspect 6. The mesenchymal stem cell population of claim 1, wherein said enhanced therapeutic activity refers to superior activity in areas selected from one or more of the following therapeutic areas: a) angiogenic; b) antiapoptotic; c) ability to stimulate endogenous regenerative cell proliferation; d) immune modulatory; e) anti-infective; and f) anti-inflammatory.

Aspect 7. The mesenchymal stem cell population of claim 1, wherein conventional mesenchymal stem cells are placenta derived CD90 and CD105 expressing cells that lack expression of CD34 and CD45.

Aspect 8. The mesenchymal stem cell of claim 6, wherein said angiogenic activity is ability to create formation of new blood vessels.

Aspect 9. The mesenchymal stem cell of claim 1, wherein said placental stem cells is cultured together with another mesenchymal stem cells, wherein said another mesenchymal stem cell has been transfected with a gene enhancing therapeutic activity of said placental mesenchymal stem cell.

Aspect 10. The mesenchymal stem cell of claim 9, wherein said another mesenchymal stem cell transferred with said gene enhancing therapeutic activity of said placental mesenchymal stem cell is selected from a group comprising of: embryonic stem cells, cord blood stem cells, stem cells, bone marrow stem cells, amniotic fluid stem cells, neuronal stem cells, circulating peripheral blood stem cells, germinal stem cells, adipose tissue derived stem cells, exfoliated teeth derived stem cells, hair follicle stem cells, amnionic membrane stem cells, dermal stem cells, parthenogenically derived stem cells, reprogrammed stem cells and side population stem cells.

Aspect 11. The mesenchymal stem cell of claim 10, wherein said genes transfected to enhance therapeutic activity are selected from a group comprising of: IMP (inosine monophosphate) dehydrogenase 2 (IMPDH2); inc finger protein 151 (pHZ-67) (ZNF151); inc finger protein, C2H2, rapidly turned over (ZNF20); inducible poly(A)-binding protein (IPABP); inducible protein (Hs.80313); inhibitor of DNA binding 2, dominant negative helix-loop-helix protein (1D2); inhibitor of kappa light polypeptide gene enhancer in B-cells, kinase complex-associated protein (IKBKAP); inositol 1,3,4-trisphosphate 5/6-kinase; inositol 1,4,5 trisphosphate receptor type 1 (ITPR1); inositol 1,4,5-trisphosphate 3-kinase B (ITPKB); inositol monophosphatase; inositol polyphosphate-5-phosphatase, 145 kD (INPP5D); Ins(1,3,4,5)P4-binding protein; insulin (INS); insulin-like growth factor 2 receptor (IGF2R); integral membrane protein 1 (ITM1); integral membrane protein 2C (ITM2C); integral membrane protein Tmp21-I (p23); integrin beta 4 binding protein (ITGB4BP); integrin, alpha 2b (platelet glycoprotein Ilb of Ilb/Illa complex, antigen CD41B) (ITGA2B); integrin, alpha 5 (fibronectin receptor, alpha polypeptide) (ITGA5); integrin, alpha L (antigen CD11A (p180), lymphocyte function-associated antigen 1; alpha polypeptide) (ITGAL); integrin, alpha M (complement componentreceptor 3, alpha; also known as CD11b (p170), macrophage antigen alpha polypeptide) (ITGAM); integrin, alpha X (antigen CD11C (p150), alpha polypeptide) (ITGAX); integrin, beta 1 (fibronectin receptor, beta polypeptide, antigen CD29 includes MDF2 MSK12) (ITGB1); integrin, beta 2 (antigen CD18 (p95), lymphocyte function-associated antigen 1; macrophage antigen 1 (mac-1) beta subunit) (ITGB2); integrin, beta 7 (ITGB7); Integrin-linked kinase (ILK); intercellular adhesion molecule 1 (CD54), human rhinovirus receptor (ICAM1); intercellular adhesion molecule 2 (ICAM2); intercellular adhesion molecule 3 (ICAM3); intercellular adhesion molecule 4, Landsteiner-Wiener blood group (ICAM4); Interferon consensus sequence binding protein 1 (ICSBP1); interferon regulatory factor 2 (IRF2); interferon regulatory factorl (IRF1); interferon regulatory factor5 (IRF5); interferon, gamma-inducible protein 16 (IFI16); interferon, gamma-inducible protein 30 (IFI30); interferon-induced protein 17 (IFI17); interferon-induced protein 54 (IFI54); interferon-inducible (1-8D); interferon-inducible (1-8U); interferon-related developmental regulator 1 (IFRD1); interferon-stimulated transcription factor 3, gamma (48 kD) (ISGF3G); interleukin 1 receptor, type II (IL1R2); Interleukin 10 receptor, beta (1.10RB); interleukin 12 receptor, beta 1 (IL12RB1); interleukin 13 receptor, alpha 1 (IL13RA1); interleukin 16 (lymphocyte chemoattractant factor) (IL16); interleukin 18 receptor 1 (IL18R1); interleukin 2 receptor, beta (IL2RB); interleukin 2 receptor, gamma (severe combined immunodeficiency) (IL2RG); interleukin 4 receptor (IL4R); interleukin 6 receptor (IL6R); interleukin 6 signal transducer (gp130, oncostatin M receptor) (IL6ST); interleukin 7 receptor (IL7R); interleukin 8 (IL8); interleukin 8 receptor alpha (IL8RA); interleukin 8 receptor, beta (IL8RB); interleukin enhancer binding factor 2, 45 kD (ILF2); interleukin enhancer binding factor 3, 90 kD (ILF3); interleukin-1 receptor-associated kinase 1 (IRAK1); interleukin-10 receptor, alpha (IL10RA); interleukin-11 receptor, and alpha (IL11RA).

Aspect 12. The mesenchymal stem cell of claim 1, wherein said mesenchymal stem cells with placental activity is selected based on expression of one or more genes selected from a group of genes below, compared to conventional mesenchymal stem cells: calnexin (CANX); calpain, large polypeptide L1 (CAPN1); calpain, large polypeptide L2 (CANP2); calpain, small polypeptide (CAPN4); calpastatin (CAST); Calponin 2; calponin 2 (CNN2); calumenin (CALU); cAMP response element-binding protein CRE-Bpa (H_GS165L15.1); cAMP-dependent protein kinase type II (Ht31); canicular multispecific organic anion transporter (CMOAT2); capping protein (actin filament) muscle Z-line, alpha 1 (CAPZA1); capping protein (actin filament) muscle Z-line, alpha 2 (CAPZA2); capping protein (actin filament) muscle Z-line, beta (CAPZB); capping protein (actin filament), gelsolin-like (CAPG); carbamoyl-phosphate synthetase 2, aspartate transcarbamylase, and dihydroorotase (CAD); carbonic anhydrase V, mitochondrial (CA5); carboxypeptidase D (CPD); cardiac beta-myosin heavy chain; carnitine/acylcarnitine translocase (CACT); Cas-Br-M (murine) ecotropic retroviral transforming sequence (cb1); casein kinase 1, alpha 1 (CSNK1A1); casein kinase 2, alpha 1 polypeptide (CSNK2A1); casein kinase I gamma 3L (CSNK1G3L); CASP8 and FADD-like apoptosis regulator (CFLAR); caspase 1, apoptosis-related cysteine protease (interleukin 1, beta, convertase) (CASP1); caspase 10, apoptosis-related cysteine proteas (CASP10); caspase 3, apoptosis-related cysteine protease (CASP3); caspase 4, apoptosis-related cysteine protease (CASP4); caspase 5, apoptosis-related cysteine protease (CASP5); caspase 8, apoptosis-related cysteine protease (CASP8); caspase 9, apoptosis-related cysteine protease (CASP9); catalase (CAT); catechol-O-methyltransferase (COMT); catenin (cadherin-associated protein), alpha 1 (102 kD) (CTNNA1); cathelicidin antimicrobial peptide (CAMP); cathepsin B (CTSB); cathepsin C (CTSC); cathepsin D (lysosomal aspartyl protease) (CTSD); cathepsin E (CTSE); cathepsin G (CTSG); cathepsin S (CTSS); cathepsin W (lymphopain) (CTSW); CCAAT/enhancer binding protein (C/EBP), alpha (CEBPA); CCAAT/enhancer binding protein (C/EBP), delta (CEBPB); CCAAT-box-binding transcription factor (CBF2); CD14 antigen (CD14); CD antigen, c polypeptide (CD1C); CD2 antigen (cytoplasmic tail)-binding protein 2 (CD2BP2); CD2 antigen (p50), sheep red blood cell receptor (CD2); CD2 cytoplasmic tail-binding protein 1 (CD2BP1); CD.sub.20 antigen (CD20); CD20 receptor (S7); CD22 antigen (CD22); CD24 signal transducer; CD33 antigen (gp67) (CD33); CD33 antigen-like 2; CD36 antigen (collagen type I receptor, thrombospondin receptor) (CD36); CD37 antigen (CD37); CD38 alt; CD39 antigen (CD39); CD3D antigen, delta polypeptide (TiT3 complex) (CD3D); CD3E antigen, epsilon polypeptide (TiT3 complex) (CD3E); CD3G antigen, gamma polypeptide (TiT3 complex) (CD3G); CD3Z antigen, zeta polypeptide (TiT3 complex) (CD3Z); CD3-zeta (clone pBS NK1); CD4 antigen (p55) (CD4); CD44 antigen (homing function and Indian blood group system (CD44); CD48 antigen (B-cell membrane protein) (CD48); CD53 antigen (CD53); CD63 antigen (melanoma 1 antigen) (CD63); CD68 antigen (CD68); CD74 antigen (invariant polypeptide of major histocompatibility complex, class II antigen-associated) (CD74); CD79A antigen (immunoglobulin-associated alpha) (CD79A); CD79B antigen (immunoglobulin-associated beta) (CD79B); CD8 antigen, alpha polypeptide (p32) (CD8A); CD8 antigen, beta polypeptide 1 (p37) (CD8B1); CD81 antigen (target of antiproliferative antibody 1 (CD81).

Aspect 13. The mesenchymal stem cell of claim 6, wherein said enhanced angiogenic activity is associated with ability to produce in absence of stimulation cytokines selected from a group comprising of: a) VEGF; b) FGF-alpha; b) FGF-beta; c) FGF-5; d) HGF; e) PDGF; f) IGF; and g) EGF.

Aspect 14. The mesenchymal stem cell of claim 6, wherein said enhanced angiogenic activity is associated with ability to produce cytokines following activation selected from a group comprising of: a) VEGF; b) FGF-alpha; b) FGF-beta; c) FGF-5; d) HGF; e) PDGF; f) IGF; and g) EGF.

Aspect 15. The mesenchymal stem cell of claim 14, wherein said stimulation is exposure to an inflammatory signal.

Aspect 16. The mesenchymal stem cell of claim 15, wherein said inflammatory signal is an activator of a toll like receptor.

Aspect 17. The mesenchymal stem cell of claim 16, wherein said toll like receptor is TLR-1.

Aspect 18. The mesenchymal stem cell of claim 17, wherein said activator of TLR-1 is Pam3CSK4.

Aspect 19. The mesenchymal stem cell of claim 16, wherein said toll like receptor is TLR-2.

Aspect 20. The mesenchymal stem cell of claim 19, wherein said activator of TLR-2 is H KLM.

Aspect 21. The mesenchymal stem cell of claim 16, wherein said toll like receptor is TLR-3.

Aspect 22. The mesenchymal stem cell of claim 21, wherein said activator of TLR-3 is Poly:IC.

Aspect 23. The mesenchymal stem cell of claim 16, wherein said toll like receptor is TLR-4.

Aspect 24. The mesenchymal stem cell of claim 23, wherein said activator of TLR-4 is LPS.

Aspect 25. The mesenchymal stem cell of claim 23, wherein said activator of TLR-4 is Buprenorphine.

Aspect 26. The mesenchymal stem cell of claim 23, wherein said activator of TLR-4 is Carbamazepine.

Aspect 27. The mesenchymal stem cell of claim 23, wherein said activator of TLR-4 is Fentanyl.

Aspect 28. The mesenchymal stem cell of claim 23, wherein said activator of TLR-4 is Levorphanol.

Aspect 29. The mesenchymal stem cell of claim 23, wherein said activator of TLR-4 is Methadone.

Aspect 30. The mesenchymal stem cell of claim 23, wherein said activator of TLR-4 is Cocaine.

Aspect 31. The mesenchymal stem cell of claim 23, wherein said activator of TLR-4 is Morphine.

Aspect 32. The mesenchymal stem cell of claim 23, wherein said activator of TLR-4 is Oxcarbazepine.

Aspect 33. The mesenchymal stem cell of claim 23, wherein said activator of TLR-4 is Oxycodone.

Aspect 34. The mesenchymal stem cell of claim 23, wherein said activator of TLR-4 is Pethidine.

Aspect 35. The mesenchymal stem cell of claim 23, wherein said activator of TLR-4 is Glucuronoxylomannan from Cryptococcus.

Aspect 36. The mesenchymal stem cell of claim 23, wherein said activator of TLR-4 is Morphine-3-glucuronide.

Aspect 37. The mesenchymal stem cell of claim 23, wherein said activator of TLR-4 is lipoteichoic acid.

Aspect 38. The mesenchymal stem cell of claim 23, wherein said activator of TLR-4 is β-defensin 2.

Aspect 39. The mesenchymal stem cell of claim 23, wherein said activator of TLR-4 is small molecular weight hyaluronic acid.

Aspect 40. The mesenchymal stem cell of claim 23, wherein said activator of TLR-4 is fibronectin EDA.

Aspect 41. The mesenchymal stem cell of claim 23, wherein said activator of TLR-4 is snapin.

Aspect 42. The mesenchymal stem cell of claim 23, wherein said activator of TLR-4 is tenascin C.

Aspect 43. The mesenchymal stem cell of claim 16, wherein said toll like receptor is TLR-5.

Aspect 44. The mesenchymal stem cell of claim 43, wherein said activator of TLR-5 is flagellin.

Aspect 45. The mesenchymal stem cell of claim 16, wherein said toll like receptor is TLR-6.

Aspect 46. The mesenchymal stem cell of claim 45, wherein said activator of TLR-6 is FSL-1.

Aspect 47. The mesenchymal stem cell of claim 16, wherein said toll like receptor is TLR-7.

Aspect 48. The mesenchymal stem cell of claim 47, wherein said activator of TLR-7 is imiquimod.

Aspect 49. The mesenchymal stem cell of claim 16, wherein said toll like receptor of TLR-8.

Aspect 50. The mesenchymal stem cell of claim 49, wherein said activator of TLR8 is ssRNA40/LyoVec.

Aspect 51. The mesenchymal stem cell of claim 16, wherein said toll like receptor of TLR-9.

Aspect 52. The mesenchymal stem cell of claim 51, wherein said activator of TLR-9 is a CpG oligonucleotide.

Aspect 53. The mesenchymal stem cell of claim 52, wherein said activator of TLR-9 is ODN2006.

Aspect 54. The mesenchymal stem cell of claim 52, wherein said activator of TLR-9 is Agatolimod.

Aspect 55. The mesenchymal stem cell of claim 6, wherein said angiogenic activity is quantified by production of an angiogenic factor, said angiogenic factor selected from a group comprising of: angiogenic polypeptide is selected from a group comprising of: activin A, adrenomedullin, aFGF, ALK1, ALKS, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, collagen receptors .alpha.sub.1.beta.sub.1 and .alpha.sub.2.beta.sub.1, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin and fibronectin receptor .alpha.5.beta.1, Factor X, HB-EGF, HBNF, HGF, HUAF, heart derived inhibitor of vascular cell proliferation, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alphatransferrin, thrombospondin, urokinase, VEGF-A, VEGF-B, VEGF-C, VEGF-D, VEGF-E, VEGF, VEGF.sub.164, VEGI, EG-VEGF.

Aspect 56. The mesenchymal stem cell of claim 6, wherein said anti-apoptotic activities comprise of augmenting bcl-2 expression in an adjacent cell.

Aspect 57. The mesenchymal stem cell of claim 6, wherein said anti-apoptotic activities comprise of augmenting bcl-xL expression in an adjacent cell.

Aspect 58. The mesenchymal stem cell of claim 6, wherein said anti-apoptotic activities comprise of augmenting XIAP expression in an adjacent cell.

Aspect 59. The mesenchymal stem cell of claim 6, wherein said anti-apoptotic activities comprise of reducing BAX expression in an adjacent cell.

Aspect 60. The mesenchymal stem cell of claim 6, wherein said anti-apoptotic activities comprise of reducing BAD expression in an adjacent cell.

Aspect 61. The mesenchymal stem cell of claim 6, wherein said anti-apoptotic activities comprise of reducing bcl-x expression in an adjacent cell.

Aspect 62. The mesenchymal stem cell of claim 6, wherein said anti-apoptotic activities comprise of augmenting bcl-2 expression in a distant cell.

Aspect 63. The mesenchymal stem cell of claim 6, wherein said anti-apoptotic activities comprise of augmenting bcl-2 expression in a distant cell through production of soluble factors.

Aspect 64. The mesenchymal stem cell of claim 6, wherein said anti-apoptotic activities comprise of augmenting bcl-xL expression in a distant cell.

Aspect 65. The mesenchymal stem cell of claim 6, wherein said anti-apoptotic activities comprise of augmenting bcl-xL expression in a distant cell through production of soluble factors.

Aspect 66. The mesenchymal stem cell of claim 6, wherein said anti-apoptotic activities comprise of augmenting XIAP expression in a distant cell.

Aspect 67. The mesenchymal stem cell of claim 6, wherein said anti-apoptotic activities comprise of augmenting XIAP expression in a distant cell through production of soluble factors.

Aspect 68. The mesenchymal stem cell of claim 6, wherein said anti-apoptotic activities comprise of reducing BAX expression in a distant cell.

Aspect 70. The mesenchymal stem cell of claim 6, wherein said anti-apoptotic activities comprise of reducing BAX expression in a distant cell through production of soluble factors.

Aspect 71. The mesenchymal stem cell of claim 6, wherein said anti-apoptotic activities comprise of reducing BAD expression in a distant cell.

Aspect 72. The mesenchymal stem cell of claim 6, wherein said anti-apoptotic activities comprise of reducing BAD expression in a distant cell through production of soluble factors.

Aspect 73. The mesenchymal stem cell of claim 6, wherein said anti-apoptotic activities comprise of reducing bcl-x expression in a distant cell.

Aspect 74. The mesenchymal stem cell of claim 6, wherein said anti-apoptotic activities comprise of reducing bcl-x expression in a distant cell through production of soluble factors.

Aspect 75. The mesenchymal stem cell of claim 6, wherein ability to stimulate endogenous regenerative cell proliferation refers to cells possessing ability to self-renew found adjacent to tissues selected from a group of tissues which contain cells, said cells selected from a group comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Aspect 76. The mesenchymal stem cell of claim 6, wherein ability to stimulate endogenous regenerative cell proliferation refers to cells possessing expression of aldehyde dehydrogenase.

Aspect 77. The mesenchymal stem cell of claim 6, wherein ability to stimulate endogenous regenerative cell proliferation refers to cells possessing expression of CD133.

Aspect 78. The mesenchymal stem cell of claim 6, wherein ability to stimulate endogenous regenerative cell proliferation refers to cells possessing expression of c-kit.

Aspect 79. The mesenchymal stem cell of claim 6, wherein ability to stimulate endogenous regenerative cell proliferation refers to cells possessing expression of nanog.

Aspect 80. The mesenchymal stem cell of claim 6, wherein ability to stimulate endogenous regenerative cell proliferation refers to cardiac specific stem cells.

Aspect 81. The mesenchymal stem cell of claim 6, wherein ability to stimulate endogenous regenerative cell proliferation refers to hepatic stem cells.

Aspect 82. The mesenchymal stem cell of claim 6, wherein ability to stimulate endogenous regenerative cell proliferation refers to cells associated with neurogenesis.

Aspect 83. The mesenchymal stem cell of claim 82, wherein ability to stimulate endogenous regenerative cell proliferation refers to regenerative cells in the dentate gyrus.

Aspect 84. The mesenchymal stem cell of claim 82, wherein ability to stimulate endogenous regenerative cell proliferation refers to regenerative cells in the subventricular zone.

Aspect 85. The mesenchymal stem cell of claim 83, wherein said regenerative cells in the dentate gyrus are neurogenic subsequent to an insult.

Aspect 86. The mesenchymal stem cell of claim 84, wherein said regenerative cells in the subventricular zone are neurogenic subsequent to an insult.

Aspect 87. The mesenchymal stem cell of claim 1, wherein said cells is treated in a manner to induce a dedifferentiation cellular program.

Aspect 88. The mesenchymal stem cell of claim 87, wherein said dedifferentiation cellular program is induced by a means selected from a group comprising of: a) nuclear transfer into a cell that is substantially more undifferentiated; b) cytoplasmic transfer of cytoplasm from an undifferentiated stem cell into said cell; c) treatment of cell with a DNA methyltransferase inhibitor; d) treatment of cell with a histone deacetylase inhibitor; e) treatment of cell with a GSK-3 inhibitor; e) treatment of cells by exposure to extracellular conditions that are conducive to stimulation of dedifferentiation; and f) treatment of cells with various combination of the mentioned treatment conditions.

Aspect 89. The mesenchymal stem cell of claim 88, wherein said nuclear transfer comprises introducing nuclear material to a cell substantially enucleated, said nuclear material deriving from a host whose genetic profile is sought to be dedifferentiated.

Aspect 90. The mesenchymal stem cell of claim 88, wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

Aspect 91. The mesenchymal stem cell of claim 88, wherein said DNA demethylating agent is selected from a group comprising of: 5-azacytidine, psammaplin A, and zebularine.

Aspect 92. The mesenchymal stem cell of claim 88, wherein said histone deacetylase inhibitor is selected from a group comprising of: valproic acid, trichostatin-A, trapoxin A and depsipeptide.

Aspect 93. The mesenchymal stem cell of claim 88, wherein said undifferentiated cells are identified based on expression multidrug resistance transport protein (ABCG2) or ability to efflux intracellular dyes such as rhodamine-123 and or Hoechst 33342.

Aspect 94. The mesenchymal stem cell of claim 88, wherein said undifferentiated cells are isolated from a population of cultured cells based on expression multidrug resistance transport protein (ABCG2) or ability to efflux intracellular dyes such as rhodamine-123 and or Hoechst 33342.

Aspect 95. The mesenchymal stem cell of claim 6, wherein said immune modulatory effects are stimulation of T regulatory cell activity.

Aspect 96. The mesenchymal stem cell of claim 95, wherein said T regulatory cell activity is assessed by ability to inhibit proliferation of a conventional T cell.

Aspect 97. The mesenchymal stem cell of claim 95, wherein said T regulatory cell activity is assessed by ability to inhibit production of interferon gamma from a conventional T cells.

Aspect 98. The mesenchymal stem cell of claim 95, wherein said T regulatory cell activity is assessed by ability to inhibit production of interleukin-2 from a conventional T cells.

Aspect 99. The mesenchymal stem cell of claim 95, wherein said T regulatory cell activity is assessed by ability to inhibit production of interleukin-4 from a conventional T cells.

Aspect 100. The mesenchymal stem cell of claim 95, wherein said T regulatory cell activity is assessed by ability to inhibit production of TNF-alpha Th17 cell.

Aspect 101. The mesenchymal stem cell of claim 1, wherein said mesenchymal stem cell expresses markers selected from a group comprising of; a) oxidized low density lipoprotein receptor 1, b) chemokine receptor ligand 3; and c) granulocyte chemotactic protein.

Aspect 102. The mesenchymal stem cell of claim 101, wherein said mesenchymal stem cell expresses, relative to a human fibroblast, increased levels of interleukin 8 and reticulon 1.

Aspect 103. The mesenchymal stem cell of claim 102, wherein said mesenchymal stem cells possesses ability to induce differentiation of another stem cell into cells of at least a skeletal muscle, vascular smooth muscle, pericyte or vascular endothelium phenotype subsequent to administration in vivo into a site of inflammation.

Aspect 104. The mesenchymal stem cell of claim 103, wherein said site of inflammation is associated with increased TNF-alpha.

Aspect 105. The mesenchymal stem cell of claim 1, wherein said mesenchymal stem cell is useful for treating a disease condition selected from a group comprising of: a) neurological disease; b) inflammatory conditions; c) psychiatric disorders; d) inborn errors of metabolisms; e) vascular disease; f) cardiac disease; g) renal disease; h) hepatic disease; i) pulmonary disease; j) ocular conditions; k) gastrointestinal disorders; l) orthopedic disorders; m) dermal disorders; n) neoplasia; o) predisposition to neoplasia; p) hematopoietic disorders; q) reproductive disorders; r) gynecological disorders; s) urological disorders; t) immunological disorders; u) olfactory disorders; and v) auricular disorders.

Aspect 106. The mesenchymal stem cell of claim 105, wherein said mesenchymal stem cell is administered allogeneic to the recipient.

Aspect 107. The mesenchymal stem cell of claim 105, wherein said mesenchymal stem cell is administered with at least a 1 out of 6 HLA match to the allogeneic recipient.

Aspect 108. The mesenchymal stem cell of claim 105, wherein said inborn error of metabolism is Krabbe disease.

Aspect 109. The mesenchymal stem cell of claim 1, wherein said mesenchymal stem cells are utilized as a source of conditioned media.

Aspect 110. The mesenchymal stem cell of claim 1, wherein said mesenchymal stem cells are utilized as a source of exosomes.

Aspect 111. The mesenchymal stem cell of claim 1, wherein said mesenchymal stem cells are utilized as a source of microvesicles.

### EXAMPLE 1

### Treatment of Critical Limb Ischemia Patients with MSC Possessing EPC Properties of the Invention

Patients who matched the following inclusion criteria where allowed into the study: a) Unreconstructable arterial disease was determined by a vascular surgeon who did not participate in the study. Unreconstructable arterial disease is defined by atherocclusive lesions within the arterial tree of the extremity that due to extent or morphology are not amenable to surgical bypass or PTCA and stenting; b) Objective evidence of severe peripheral arterial disease includes an ankle brachial index (ABI) of less than 0.60, and/or a resting toe brachial index (TBI) of less than 40; c) Patients were competent to give consent; and d) No history of malignant disease except for nonmelanoma skin cancer, no suspicious findings on chest x-ray, mammography (women over age 35), Papanicolaou smear (women over age 40), a normal fecal occult blood (over age 50) and a normal prostate specific antigen (men over age 45).

Patients were injected with 10(8) cells generated as described in the specification. Injections were performed using 10 million cells per injection, with 10 injections locally at the area of failed perfusion in a 10 cm.times.10 cm area in the gastrocnemius muscle. Ankle Brachial index was measured by comparing the ankle and brachial pressure. No patients reported amputation during the study period.

Results are provided above in the table below.

TABLE 1

| | | Treatment Related Adverse | ABI | ABI |
|---|---|---|---|---|
| ID | Date of Birth | Events | Pre | 6 months |
| AS | Feb. 5, 1954 | None | .57 | .75 |
| PN | Jan. 13, 1967 | None | .55 | .57 |
| JB | Dec. 4, 1969 | None | .49 | .80 |
| NR | Mar. 15, 1964 | Mild self limiting inflammation | .58 | .71 |
| | | at injection site | | |
| SP | May 17, 1958 | None | .50 | .73 |
| ES | Apr. 2, 1970 | None | .52 | .77 |
| JS | Dec. 5, 1959 | Headache after injection | .55 | .74 |
| FG | Apr. 17, 1952 | Mild self limiting inflammation | .60 | .81 |
| | | at injection site | | |
| HS | Apr. 25, 1963 | None | .58 | .55 |
| JP | Aug. 13, 1959 | None | .58 | .69 |

### REFERENCES

1. Zhang, X. Y., et al., Lentiviral vectors for sustained transgene expression in human bone marrow-derived stromal cells. Mol Ther, 2002. 5(5 Pt 1): p. 555-65.
2. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.
3. Worsham, D. N., et al., In vivo gene transfer into adult stem cells in unconditioned mice by in situ delivery of a lentiviral vector. Mol Ther, 2006. 14(4): p. 514-24.
4. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.
5. Kallifatidis, G., et al., Improved lentiviral transduction of human mesenchymal stem cells for therapeutic intervention in pancreatic cancer. Cancer Gene Ther, 2008. 15(4): p. 231-40.
6. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.
7. McGinley, L., et al., Lentiviral vector mediated modification of mesenchymal stem cells & enhanced survival in an in vitro model of ischaemia. Stem Cell Res Ther, 2011. 2(2): p. 12.
8. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.
9. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.
10. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.
11. Yu, Y. S., et al., AKT-modified autologous intracoronary mesenchymal stem cells prevent remodeling and repair in swine infarcted myocardium. Chin Med J (Engl), 2010. 123(13): p. 1702-8.
12. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.
13. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.
14. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.
15. Sage, E. K., et al., Systemic but not topical TRAIL-expressing mesenchymal stem cells reduce tumour growth in malignant mesothelioma. Thorax, 2014. 69(7): p. 638-47.
16. Lian, W. S., et al., In vivo therapy of myocardial infarction with mesenchymal stem cells modified with prostaglandin I synthase gene improves cardiac performance in mice. Life Sci, 2011. 88(9-10): p. 455-64.
17. Maijenburg, M. W., et al., Nuclear receptors Nur77 and Nurr1 modulate mesenchymal stromal cell migration. Stem Cells Dev, 2012. 21(2): p. 228-38.
18. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.
19. Zou, D., et al., In vitro study of enhanced osteogenesis induced by HIF-1alpha-transduced bone marrow stem cells. Cell Prolif, 2011. 44(3): p. 234-43.
20. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.
21. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.
22. Yang, H. M., et al., Enhancement of the immunosuppressive effect of human adipose tissue-derived mesenchymal stromal cells through HLA-G1 expression. Cytotherapy, 2012. 14(1): p. 70-9.
23. Liang, X. J., et al., Differentiation of human umbilical cord mesenchymal stem cells into hepatocyte-like cells by hTERT gene transfection in vitro. Cell Biol Int, 2012. 36(2): p. 215-21.
24. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.
25. Jaganathan, B. G. and D. Bonnet, Human mesenchymal stromal cells senesce with exogenous OCT4. Cytotherapy, 2012. 14(9): p. 1054-63.
26. Han, S. H., et al., Effect of ectopic OCT4 expression on canine adipose tissue-derived mesenchymal stem cell proliferation. Cell Biol Int, 2014. 38(10): p. 1163-73.
27. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.
28. Kearns-Jonker, M., et al., Genetically Engineered Mesenchymal Stem Cells Influence Gene Expression in Donor Cardiomyocytes and the Recipient Heart. J Stem Cell Res Ther, 2012. S1.
29. Ma, G. L., et al., [Study of inhibiting and killing effects of transgenic LIGHT human umbilical cord blood mesenchymal stem cells on stomach cancer]. Zhonghua Wei Chang Wai Ke Za Zhi, 2012. 15(11): p. 1178-81.
30. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.
31. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.
32. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
33. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.
34. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.
35. Cao, Z., et al., Protective effects of mesenchymal stem cells with CXCR4 up-regulation in a rat renal transplantation model. PLoS One, 2013. 8(12): p. e82949.
36. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.
37. Zhu, Y., et al., Mesenchymal stem cell-based NK4 gene therapy in nude mice bearing gastric cancer xenografts. Drug Des Devel Ther, 2014. 8: p. 2449-62.
38. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.
39. Xu, W., et al., Basic fibroblast growth factor expression is implicated in mesenchymal stem cells response to light-induced retinal injury. Cell Mol Neurobiol, 2013. 33(8): p. 1171-9.
40. Li, W., et al., Bcl-2 engineered MSCs inhibited apoptosis and improved heart function. Stem Cells, 2007. 25(8): p. 2118-27.
41. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.
42. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.
43. Le Blanc, K., et al., Treatment of severe acute graft-versus-host disease with third party haploidentical mesenchymal stem cells. Lancet, 2004. 363(9419): p. 1439-41.
44. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.
45. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.
46. Reinisch, A., et al., Humanized system to propagate cord blood-derived multipotent mesenchymal stromal cells for clinical application. Regen Med, 2007. 2(4): p. 371-82.
47. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.
48. Lataillade, J. J., et al., New approach to radiation burn treatment by dosimetry-guided surgery combined with autologous mesenchymal stem cell therapy. Regen Med, 2007. 2(5): p. 785-94.
49. Seshareddy, K., D. Troyer, and M. L. Weiss, Method to isolate mesenchymal-like cells from Wharton's Jelly of umbilical cord. Methods Cell Biol, 2008. 86: p. 101-19.
50. Sensebe, L., Clinical grade production of mesenchymal stem cells. Biomed Mater Eng, 2008. 18(1 Suppl): p. S3-10.
51. Sotiropoulou, P. A., S. A. Perez, and M. Papamichail, Clinical grade expansion of human bone marrow mesenchymal stem cells. Methods Mol Biol, 2007. 407: p. 245-63.
52. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.
53. Zhang, X., et al., Cotransplantation of HLA-identical mesenchymal stem cells and hematopoietic stem cells in Chinese patients with hematologic diseases. Int J Lab Hematol, 2010. 32(2): p. 256-64.
54. Arrigoni, E., et al., Isolation, characterization and osteogenic differentiation of adipose-derived stem cells: from small to large animal models. Cell Tissue Res, 2009. 338(3): p. 401-11.
55. Grisendi, G., et al., GMP-manufactured density gradient media for optimized mesenchymal stromal/stem cell isolation and expansion. Cytotherapy, 2010. 12(4): p. 466-77.
56. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.
57. Sensebe, L., P. Bourin, and K. Tarte, Good manufacturing practices production of mesenchymal stem/stromal cells. Hum Gene Ther, 2011. 22(1): p. 19-26.
58. Capelli, C., et al., Minimally manipulated whole human umbilical cord is a rich source of clinical-grade human mesenchymal stromal cells expanded in human platelet lysate. Cytotherapy, 2011. 13(7): p. 786-801.
59. Ilic, N., et al., Manufacture of clinical grade human placenta-derived multipotent mesenchymal stromal cells. Methods Mol Biol, 2011. 698: p. 89-106.
60. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.
61. Timmins, N. E., et al., Closed system isolation and scalable expansion of human placental mesenchymal stem cells. Biotechnol Bioeng, 2012. 109(7): p. 1817-26.
62. Warnke, P. H., et al., A clinically feasible protocol for using human platelet lysate and mesenchymal stem cells in regenerative therapies. J Craniomaxillofac Surg, 2013. 41(2): p. 153-61.
63. Fekete, N., et al., GMP-compliant isolation and large-scale expansion of bone marrow-derived MSC. PLoS One, 2012. 7(8): p. e43255.
64. Hanley, P. J., et al., Manufacturing mesenchymal stromal cells for phase I clinical trials. Cytotherapy, 2013. 15(4): p. 416-22.
65. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. 15(9): p. 1086-97.
66. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.
67. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.
68. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.
69. Martins, J. P., et al., Towards an advanced therapy medicinal product based on mesenchymal stromal cells isolated from the umbilical cord tissue: quality and safety data. Stem Cell Res Ther, 2014. 5(1): p. 9.
70. ludicone, P., et al., Pathogen-free, plasma-poor platelet lysate and expansion of human mesenchymal stem cells. J Transl Med, 2014. 12: p. 28.
71. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.
72. Ikebe, C. and K. Suzuki, Mesenchymal stem cells for regenerative therapy: optimization of cell preparation protocols. Biomed Res Int, 2014. 2014: p. 951512.
73. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.
74. Swamynathan, P., et al., Are serum free and xeno-free culture conditions ideal for large scale clinical grade expansion of Wharton's jelly derived mesenchymal stem cells? A comparative study. Stem Cell Res Ther, 2014. 5(4): p. 88.
75. Vaes, B., et al., Culturing protocols for human multipotent adult stem cells. Methods Mol Biol, 2015. 1235: p. 49-58.
76. Devito, L., et al., Wharton's jelly mesenchymal stromal/stem cells derived under chemically defined animal product-free low oxygen conditions are rich in MSCA-1(+) subpopulation. Regen Med, 2014. 9(6): p. 723-32.
77. Zhu, X. Y., et al., Mesenchymal stem cells and endothelial progenitor cells decrease renal injury in experimental swine renal artery stenosis through different mechanisms. Stem Cells, 2013. 31(1): p. 117-25.
78. Lopez-Holgado, N., et al., Short-term endothelial progenitor cell colonies are composed of monocytes and do not acquire endothelial markers. Cytotherapy, 2007. 9(1): p. 14-22.
79. Achyut, B. R. and A. S. Arbab, Myeloid Derived Suppressor Cells: Fuel the Fire. Biochem Physiol, 2014. 3(3): p. e123.
80. Sander, A. L., et al., Systemic transplantation of progenitor cells accelerates wound epithelialization and neovascularization in the hairless mouse ear wound model. J Surg Res, 2011. 165(1): p. 165-70.
81. Fortini, C., et al., Circulating stem cell vary with NYHA stage in heart failure patients. J Cell Mol Med, 2011. 15(8): p. 1726-36.
82. Tardif, K., et al., A phosphorylcholine-modified chitosan polymer as an endothelial progenitor cell supporting matrix. Biomaterials, 2011. 32(22): p. 5046-55.
83. Campioni, D., et al., In vitro characterization of circulating endothelial progenitor cells isolated from patients with acute coronary syndrome. PLoS One, 2013. 8(2): p. e56377.
84. Nowak, W. N., et al., Number of circulating pro-angiogenic cells, growth factor and anti-oxidative gene profiles might be altered in type 2 diabetes with and without diabetic foot syndrome. J Diabetes Investig, 2014. 5(1): p. 99-107.
85. Obtulowicz, P., et al., Induction of Endothelial Phenotype From Wharton's Jelly-Derived MSCs and Comparison of Their Vasoprotective and Neuroprotective Potential With Primary WJ-MSCs in CA1 Hippocampal Region Ex Vivo. Cell Transplant, 2016. 25(4): p. 715-27.
86. Rohde, E., et al., Blood monocytes mimic endothelial progenitor cells. Stem Cells, 2006. 24(2): p. 357-67.
87. Bagley, R. G., et al., Pericytes and endothelial precursor cells: cellular interactions and contributions to malignancy. Cancer Res, 2005. 65(21): p. 9741-50.
88. Finney, M. R., et al., Direct comparison of umbilical cord blood versus bone marrow-derived endothelial precursor cells in mediating neovascularization in response to vascular ischemia. Biol Blood Marrow Transplant, 2006. 12(5): p. 585-93.
89. Neumuller, J., et al., Immunological and ultrastructural characterization of endothelial cell cultures differentiated from human cord blood derived endothelial progenitor cells. Histochem Cell Biol, 2006. 126(6): p. 649-64.
90. Nguyen, V. A., et al., Endothelial cells from cord blood CD133+CD34+ progenitors share phenotypic, functional and gene expression profile similarities with lymphatics. J Cell Mol Med, 2009. 13(3): p. 522-34.
91. Kamei, N., et al., Lnk deletion reinforces the function of bone marrow progenitors in promoting neovascularization and astrogliosis following spinal cord injury. Stem Cells, 2010. 28(2): p. 365-75.
92. Kuliczkowski, W., et al., Endothelial progenitor cells and left ventricle function in patients with acute myocardial infarction: potential therapeutic considertions. Am J Ther, 2012. 19(1): p. 44-50.
93. Damaghi, M. and R. Gillies, Phenotypic changes of acid adapted cancer cells push them toward aggressiveness in their evolution in the tumor microenvironment. Cell Cycle, 2016: p. 0.
94. Lobo, R. C., et al., Glucose Uptake and Intracellular pH in a Mouse Model of Ductal Carcinoma In situ (DCIS) Suggests Metabolic Heterogeneity. Front Cell Dev Biol, 2016. 4: p. 93.
95. An, S., et al., Amino Acid Metabolism Abnormity and Microenvironment Variation Mediated Targeting and Controlled Glioma Chemotherapy. Small, 2016.
96. Avnet, S., et al., Altered pH gradient at the plasma membrane of osteosarcoma cells is a key mechanism of drug resistance. Oncotarget, 2016.
97. Huang, S., et al., Acidic extracellular pH promotes prostate cancer bone metastasis by enhancing PC-3 stem cell characteristics, cell invasiveness and VEGF-induced vasculogenesis of BM-EPCs. Oncol Rep, 2016. 36(4): p. 2025-32.
98. Carnero, A. and M. Lleonart, The hypoxic microenvironment: A determinant of cancer stem cell evolution. Bioessays, 2016. 38 Suppl 1: p. S65-74.
99. Pellegrini, P., et al., Tumor acidosis enhances cytotoxic effects and autophagy inhibition by salinomycin on cancer cell lines and cancer stem cells. Oncotarget, 2016. 7(24): p. 35703-35723.
100. Bohme, I. and A. K. Bosserhoff, Acidic tumor microenvironment in human melanoma. Pigment Cell Melanoma Res, 2016. 29(5): p. 508-23.
101. Gentric, G., V. Mieulet, and F. Mechta-Grigoriou, Heterogeneity in Cancer Metabolism: New Concepts in an Old Field. Antioxid Redox Signal, 2016.
102. Ge, Y., et al., Preferential extension of short telomeres induced by low extracellular pH. Nucleic Acids Res, 2016. 44(17): p. 8086-96.
103. Quail, D.F., et al., The tumor microenvironment underlies acquired resistance to CSF-1R inhibition in gliomas. Science, 2016. 352(6288): p. aad3018.
104. Li, X., et al., The altered glucose metabolism in tumor and a tumor acidic microenvironment associated with extracellular matrix metalloproteinase inducer and monocarboxylate transporters. Oncotarget, 2016. 7(17): p. 23141-55.
105. Zhao, C., et al., Tumor Acidity-Induced Sheddable Polyethylenimine-Poly(trimethylene carbonate)/DNA/Polyethylene Glycol-2,3-Dimethylmaleicanhydride Ternary Complex for Efficient and Safe Gene Delivery. ACS Appl Mater Interfaces, 2016. 8(10): p. 6400-10.
106. Riemann, A., et al., Acidosis Promotes Metastasis Formation by Enhancing Tumor Cell Motility. Adv Exp Med Biol, 2016. 876: p. 215-20.
107. Ravishankar, B., et al., The amino acid sensor GCN2 inhibits inflammatory responses to apoptotic cells promoting tolerance and suppressing systemic autoimmunity. Proc Natl Acad Sci USA, 2015. 112(34): p. 10774-9.
108. Walls, J., L. Sinclair, and D. Finlay, Nutrient sensing, signal transduction and immune responses. Semin Immunol, 2016.
109. Munn, D. H., et al., GCN2 kinase in T cells mediates proliferative arrest and anergy induction in response to indoleamine 2,3-dioxygenase. Immunity, 2005. 22(5): p. 633-42.
110. Ishimura, R., et al., Activation of GCN2 kinase by ribosome stalling links translation elongation with translation initiation. Elife, 2016. 5.
111. Lageix, S., et al., Enhanced interaction between pseudokinase and kinase domains in Gcn2 stimulates elF2alpha phosphorylation in starved cells. PLoS Genet, 2014. 10(5): p. e1004326.
112. Metz, R., et al., IDO inhibits a tryptophan sufficiency signal that stimulates mTOR: A novel IDO effector pathway targeted by D-1-methyl-tryptophan. Oncoimmunology, 2012. 1(9): p. 1460-1468.
113. Manlapat, A. K., et al., Cell-autonomous control of interferon type I expression by indoleamine 2,3-dioxygenase in regulatory CD19+ dendritic cells. Eur J Immunol, 2007. 37(4): p. 1064-71.
114. Sharma, M. D., et al., Plasmacytoid dendritic cells from mouse tumor-draining lymph nodes directly activate mature Tregs via indoleamine 2,3-dioxygenase. J Clin Invest, 2007. 117(9): p. 2570-82.
115. Forouzandeh, F., et al., Differential immunosuppressive effect of indoleamine 2,3-dioxygenase (IDO) on primary human CD4+ and CD8+ T cells. Mol Cell Biochem, 2008. 309(1-2): p. 1-7.
116. Wek, R. C., H. Y. Jiang, and T. G. Anthony, Coping with stress: eIF2 kinases and translational control. Biochem Soc Trans, 2006. 34(Pt 1): p. 7-11.
117. Xia, X.J., et al., Autophagy mediated by arginine depletion activation of the nutrient sensor GCN2 contributes to interferon-gamma-induced malignant transformation of primary bovine mammary epithelial cells. Cell Death Discov, 2016. 2: p. 15065.
118. Xia, X., et al., Arginine Supplementation Recovered the IFN-gamma-Mediated Decrease in Milk Protein and Fat Synthesis by Inhibiting the GCN2/eIF2alpha Pathway, Which Induces Autophagy in Primary Bovine Mammary Epithelial Cells. Mol Cells, 2016. 39(5): p. 410-7.
119. Vynnytska-Myronovska, B. O., et al., Arginine starvation in colorectal carcinoma cells: Sensing, impact on translation control and cell cycle distribution. Exp Cell Res, 2016. 341(1): p. 67-74.
120. Marion, V., et al., Arginine deficiency causes runting in the suckling period by selectively activating the stress kinase GCN2. J Biol Chem, 2011. 286(11): p. 8866-74.
121. Rodriguez, P. C., D. G. Quiceno, and A. C. Ochoa, L-arginine availability regulates T-lymphocyte cell-cycle progression. Blood, 2007. 109(4): p. 1568-73.
122. Bunpo, P., et al., The eIF2 kinase GCN2 is essential for the murine immune system to adapt to amino acid deprivation by asparaginase. J Nutr, 2010. 140(11): p. 2020-7.
123. Bunpo, P., et al., GCN2 protein kinase is required to activate amino acid deprivation responses in mice treated with the anti-cancer agent L-asparaginase. J Biol Chem, 2009. 284(47): p. 32742-9.
124. Balasubramanian, M. N., E. A. Butterworth, and M. S. Kilberg, Asparagine synthetase: regulation by cell stress and involvement in tumor biology. Am J Physiol Endocrinol Metab, 2013. 304(8): p. E789-99.
125. Wilson, G. J., et al., The eukaryotic initiation factor 2 kinase GCN2 protects against hepatotoxicity during asparaginase treatment. Am J Physiol Endocrinol Metab, 2013. 305(9): p. E1124-33.
126. Ye, J., et al., GCN2 sustains mTORC1 suppression upon amino acid deprivation by inducing Sestrin2. Genes Dev, 2015. 29(22): p. 2331-6.
127. Reinert, R. B., et al., Role of glutamine depletion in directing tissue-specific nutrient stress responses to L-asparaginase. J Biol Chem, 2006. 281(42): p. 31222-33.
128. Drogat, B., et al., Acute L-glutamine deprivation compromises VEGF-a upregulation in A549/8 human carcinoma cells. J Cell Physiol, 2007. 212(2): p. 463-72.

1. A method of generating mesenchymal stem cells possessing properties of endothelial progenitor cells comprising:
(i) isolating a mammalian cellular population;
(ii) enriching said mammalian cellular population for a subpopulation of cells expressing a CD45− phenotypic profile;
(iii) enriching said CD45− cells for a subpopulation of cells which express a CD56+ phenotypic profile; and
(iv) isolating the subpopulation of said CD56+ cells for a subpopulation of cells which express a phenotypic profile selected from the group consisting of: CD31+ and CD45−.

2. The method of claim 1, wherein said isolated subpopulation of cells which express a phenotypic profile selected from the group consisting of: CD31+ and CD45− are plastic adherent.

3. The method of claim 2, wherein said CD31+ cell population of cells is derived from fetal cells of the placenta.

4. The method of claim 2, wherein said CD31− and CD56+ cell populations are derived from fetal cells of the placenta.

5. The method of claim 2, wherein said CD31−, CD56+ cell, and CD45− cell populations are derived from fetal cells of the placenta.

6. The method of claim 2, wherein said endothelial progenitor cell property is ability to stimulate angiogenic activity.

7. The method of claim 6, wherein said angiogenic activity is associated with the ability to produce, in absence of stimulation cytokines, a factor selected from the group consisting of: a) VEGF, b) FGF-alpha, b) FGF-beta, c) FGF-5, d) HGF, e) PDGF, f) IGF, and g) EGF.

8. The method of claim 6, wherein said enhanced angiogenic activity is associated with ability to produce cytokines following activation selected from the group consisting of: a) VEGF, b) FGF-alpha, b) FGF-beta, c) FGF-5, d) HGF, e) PDGF, f) IGF, and g) EGF.

9. The method of claim 8, wherein said activation is exposure to an inflammatory signal.

10. The method of claim 9, wherein said inflammatory signal is an activator of a toll like receptor.

11. The method of claim 10, wherein said toll like receptor is TLR-1.

12. The method of claim 11, wherein said activator of TLR-1 is Pam3CSK4.

13. The method of claim 10, wherein said toll like receptor is TLR-2.

14. The method of claim 13, wherein said activator of TLR-2 is HKLM.

15. The method of claim 10, wherein said toll like receptor is TLR-3.

16. The method of claim 15, wherein said activator of TLR-3 is Poly:IC.

17. The method of claim 10, wherein said toll like receptor is TLR-4.

18. The method of claim 17, wherein said activator of TLR-4 is LPS.

19. The method of claim 17, wherein said activator of TLR-4 is Buprenorphine.

20. The method of claim 17, wherein said activator of TLR-4 is Carbamazepine.
